(19) United States
(12) Patent Application Publication
(10) Pub. No.: US 2008/0021424 A1
(43) Pub. Date: Jan. 24, 2008

(54) Side seams for absorbent articles

(76) Inventor: Edward Erdman, West Chester, PA (US)

Correspondence Address: RATNERPRESTIA, P O BOX 980, VALLEY FORGE, PA, 19482-0980, US

(21) Appl. No.: 11/239,939

(22) Filed: Sep. 30, 2005

(51) Int. Cl. A61F 13/15

(52) U.S. Cl. 604/365; 604/358; 604/385.01

(57) ABSTRACT

An absorbent article is provided that includes an absorbent core disposed between a body-facing surface and a garment-facing surface wherein the two surfaces are joined to form side seams in a bonding pattern. The bonding pattern of the surfaces at the side seams increases the softness and flexibility of the side seam while maintaining a high side seam tear strength.

### BACKGROUND

1. Technical Field

The present disclosure generally relates to absorbent articles, and more particularly to absorbent articles having a side seam structure including a bonding pattern that has an advantageous configuration that improves tear strength, softness and flexibility.

2. Description of the Related Art

Absorbent articles such as, for example, disposable diapers, adult incontinent pads, sanitary napkins, pantiliners, incontinent garments and the like are generally worn, in cooperation with garments and disposed against a body surface by infants or adult incontinent individuals. The absorbent article is employed to collect and absorb body fluid discharge, such as, for example, blood, menses, urine, aqueous body fluids, mucus, cellular debris and other forms of body discharge. For example, the absorbent article may be disposed between the legs of an individual adjacent a crotch area. The absorbent article is positioned with a garment and drawn into engagement with a body surface of the crotch area to collect body discharge.

Infants, toddlers, and other incontinent individuals wear disposable absorbent articles such as diapers and training pants, which include “pull-on” or “pant-type” garments having fixed sides, single-use diaper garments including front and back portions joined, usually by an adhesive, when the diaper is initially donned, and hybrid garments which are donned like a diaper garment but are designed to be removed and replaced like a pant-type garment.

As is known, absorbent articles typically include a fluid permeable coverstock for engaging the body surface, a fluid impermeable backsheet and an absorbent core supported therebetween. The backsheet serves as a moisture barrier to prevent fluid leakage to the garment. The absorbent core usually includes a liquid retention material that faces the body surface. The absorbent core can include, for example, loosely formed cellulosic fibers, such as, for example, wood pulp, rayon, or cotton for acquiring and storing body discharge. The absorbent core may also include tissue wrapping or wadding, and/or super absorbent polymer.

Some absorbent article designs typically incorporate elastic elements in the waist, stomach, and leg areas for improving the fit, aesthetics and leakage resistance. The waist and stomach elastic elements increase the flexibility of the absorbent article, allowing the same article to accommodate a greater range of body sizes. In addition, they make the article more form-fitting for the wearer.

In a typical configuration, the topsheet and backsheet are joined to form the side seams of the absorbent article and enclose the absorbent core. The elastic elements such as the waist band and leg bands are often enclosed between the topsheet and backsheet. Thus, the side seams normally define the edges of the absorbent article and rest against the wearer's skin. The commonality among all types of absorbent devices is their disposition in or against highly mobile areas of the body. The side seams of some current absorbent article designs can be rigid and inflexible, disadvantageously causing discomfort to the wearer.

It would be, therefore, desirable to overcome the disadvantages and drawbacks of the prior art by providing an absorbent article having a side seam structure including a configuration that improves the tear strength, softness, and/or flexibility of the side seams. Desirably, the side seams, which typically form the outer boundary of the article and rest against the wearer's skin, should be as soft and flexible as possible and have a high tear strength such that the article remains intact under normal wear conditions. Most desirably, such side seam structures prevent leakage. It is contemplated that the absorbent article is low cost and easy and efficient to manufacture.

### SUMMARY

Accordingly, the present disclosure provides an absorbent article that has a side seam structure including a configuration that improves strength, softness, and flexibility properties of absorbent article. Desirably, the side seam structure prevents leakage. The absorbent article is low cost and easy and efficient to manufacture. Objects and advantages of the present disclosure are set forth in part herein and in part will be obvious therefrom, or may be learned by practice of the present disclosure that is realized and attained by the instrumentalities and combinations pointed out in the appended claims for the devices and methods of the present disclosure consisting of its constituent parts, constructions, arrangements, combinations, steps and improvements herein shown and described.

In one particular embodiment, in accordance with the principles of the present disclosure, an absorbent article is provided that comprises an absorbent core disposed between a body-facing surface and a garment-facing surface. A side seam is formed by joining the opposing lateral edges of the body-facing surface of the front waist area of the article with the opposing lateral edges of the rear waist area with a bonding pattern. The bonding pattern comprises a plurality of rows of bonding points along the side seam. The rows are disposed parallel with the side seam. In preferred embodiments, the bonding points are circles, ovals, or ellipses, or a combination thereof. It is contemplated that the side seam comprises two, three, four, five, or more rows of bonding points.

In another embodiment, the bonding points are circles that have a diameter of about 0.5 mm to about 3.0 mm, but preferably about 1.0 mm to about 2.0 mm. The rows of bonding points may be horizontally separated by a distance equal to about 0.10 to about 1.25 times the diameter of the circles. Preferably, the bonding points are separated by a distance equal to about 0.25 to about 0.75 times the diameter of the circles. It is contemplated that the rows are horizontally separated by the same or different distances.

In another preferred embodiment, the bonding points are vertically offset in adjacent rows and the offset is equal to about 0.25 to about 0.50 times the diameter or vertical dimension of the bonding points.

In another preferred embodiment, at least one row comprises fewer bonding points than the laterally adjacent row. Preferably, the row comprises between about a quarter and three quarters as many bonding points as the laterally adjacent row. More preferably, the row comprises about half as many bonding points as the laterally adjacent row. In the most preferred embodiment, the row comprising fewer bonding points is the innermost row, which is closest to the front to back centerline of the article, of the side seam.

In addition to side seams comprising bonding points that are uniform in size and shape, it is contemplated that the side seam comprises bonding points having different shapes and/or different sizes.

In another embodiment, an absorbent article is provided that comprises an absorbent core disposed between a body-facing surface and a garment-facing surface, wherein a side seam is formed by joining the opposing lateral edges of the body-facing surface of the front waist area of the article with the opposing lateral edges of the rear waist area with a bonding pattern. The bonding pattern comprises a row of bonding points along the side seam. The row is disposed parallel with the side seam and the bonding points have an oval shape. The oval shape is disposed at an angle of between about 20° to about 60° from an axis perpendicular to the side seam. In preferred embodiments, the oval shape is disposed at an angle of between about 30° and about 45°

It is contemplated that the oval-shaped bonding points may have substantially the same size or substantially different sizes. In one embodiment, the bonding pattern comprises bonding points having a first oval length and bonding points having a second oval length, wherein the second oval length is between about 0.25 and about 0.75 times the first oval length.

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1 is a perspective view of an absorbent article in accordance with the principles of the present disclosure;

FIG. 1A is an enlarged perspective view of the indicated area of detail of the absorbent article shown in FIG. 1;

FIG. 2 is a schematic view of a side seam bonding pattern of the absorbent article shown in FIG. 1;

FIG. 3 is a schematic view of an alternate configuration of the side seam bonding pattern shown in FIG. 2;

FIG. 4 is a schematic view of another alternate configuration of the side seam bonding pattern shown in FIG. 2;

FIG. 5 is an enlarged schematic view of another alternate configuration of the side seam bonding pattern shown in FIG. 2;

FIG. 6 is an enlarged schematic view of another alternate configuration of the side seam bonding pattern shown in FIG. 2; and

FIG. 7 is an enlarged schematic view another alternate configuration of the side seam bonding pattern in accordance with the principles of the present disclosure.

### DETAILED DESCRIPTION OF THE EXEMPLARY EMBODIMENTS

The present disclosure provides an absorbent article having a side seam structure including a configuration that improves strength, softness, and flexibility of the absorbent article. The exemplary embodiments of the improved side seams are disclosed and are contemplated to be employed with absorbent articles including, for example, training pants, adult incontinent products, disposable diapers, and other incontinent articles. It is contemplated that the improved side seams have increased flexibility and softness, making them more comfortable for the wearer. Further, the improved side seams of the present disclosure maintain a high tear strength along the axis perpendicular to the direction of the side seam and maintain high bonding strength between the opposing lateral edges of the body-facing surface of the front waist area of the article with the opposing lateral edges of the rear waist area.

In the discussion that follows, the term “body-facing surface” refers to a portion of a structure that is oriented towards a body surface, and the “garment-facing surface” refers to a portion of the structure that is oriented towards a garment and is typically opposing the body-facing surface and may be referred to as such. As used herein, the term “body surface” refers to a portion of an individual's body that the absorbent article is disposed with for collecting and absorbing fluid discharge from the individual. The term “side seam” refers to the bonding or junction points that join the opposing lateral edges of the body-facing surface of the front waist area of the article with the opposing lateral edges of the rear waist area and may define regions adjacent to the other outer edges of the absorbent article, wherein the body facing surface is joined to the garment facing surface. Side seams typically form the outer edge defining the torso opening at the top of the waistband and the leg openings. For diaper-style absorbent articles, side seams also define the lateral edges of the side panels.

As used herein, the term “absorbent article,” “absorbent garment” or “garment” refers to garments that absorb and contain body waste, and more specifically, refers to garments that are placed against or in proximity to the body of the wearer to absorb and contain the various body discharges and wastes. A non-exhaustive list of examples of absorbent articles includes training pants, diapers, diaper covers, disposable diapers, feminine hygiene products and adult incontinence products. The term absorbent articles includes all variations of absorbent garments, including disposable absorbent garments that are intended to be discarded or partially discarded after a single use and unitary disposable absorbent garments that have essentially a single structure. As used herein, the term “training pants” refers to an absorbent article generally worn by children about the lower torso. The absorbent article may be disposable or partially discarded after a single use. The absorbent article can be a unitary structure or include replaceable inserts or interchangeable parts.

Absorbent articles and diapers may have a number of different constructions. In each of these constructions it is generally the case that an absorbent core is disposed between a liquid pervious, body-facing top sheet and a liquid impervious, exterior garment-facing back sheet. In some cases, one or both of the top sheet and back sheet may be shaped to form a pant-like article, such as a training pant. In other cases, the top sheet, back sheet and absorbent core may be formed as a discrete assembly that is placed on a main chassis layer and the chassis layer is shaped to form a pant-like absorbent article. The absorbent article may be provided to the consumer in the fully assembled pant-like shape or may be partially pant-like and require the consumer to take the final steps necessary to form the final pant-like shape. Training pants are typically fully formed and applied by pulling the absorbent article over a child's legs. For a diaper application, an open article is provided and wrapped about a child's waist, such as by manually fastening one or more fasteners or fastener tabs.

The following discussion includes a description of the disposable absorbent article in accordance with the present disclosure. Reference will now be made in detail to the exemplary embodiments of the disclosure, which are illustrated in the accompanying figures.

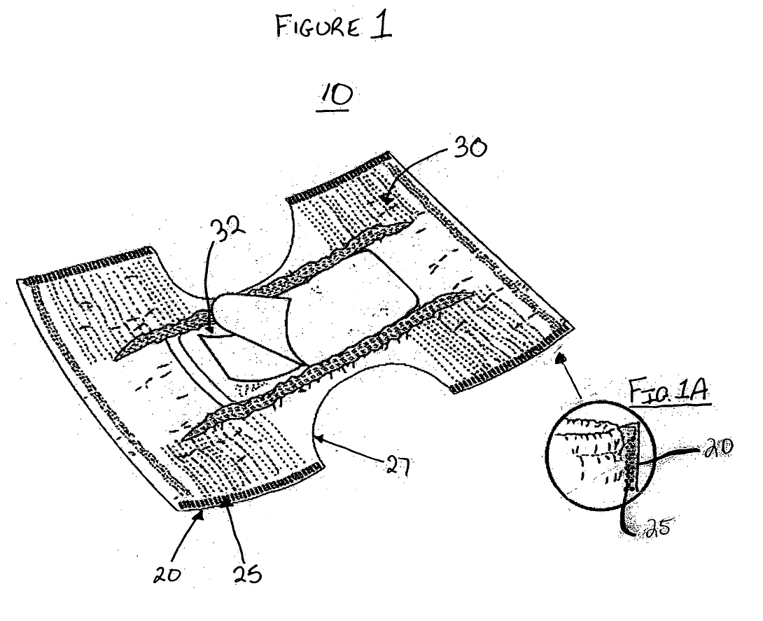

FIGS. 1 and 1A illustrate a diaper-style absorbent article 10 having a body-facing surface 30 that comprises a fluid-permeable topsheet 32. The absorbent article 10 also comprises a side seam 20 having a bonding pattern 25. Optionally, the absorbent article 10 may have a leg-opening side seam 27 consisting of the same or a different bonding pattern 25.

It is contemplated that the side seams have a bonding pattern including a plurality of rows (i.e, two, three, four, five, or more rows) of bonding points that run parallel to the side seam. The bonding pattern is configured to provide improved softness and flexibility in all folding directions. In preferred embodiments, the bonding points are round, elliptical, or oval. Bonding points having other shapes consistent with this disclosure are also contemplated. Also contemplated are bonding patterns containing a mixture of different shapes. In preferred embodiments, the bonding points are in adjacent rows offset (i.e., non-parallel) to one another. Each row need not contain the same number or shape of bonding points as any adjacent row. In one embodiment, the innermost row has fewer bonding points than another row.

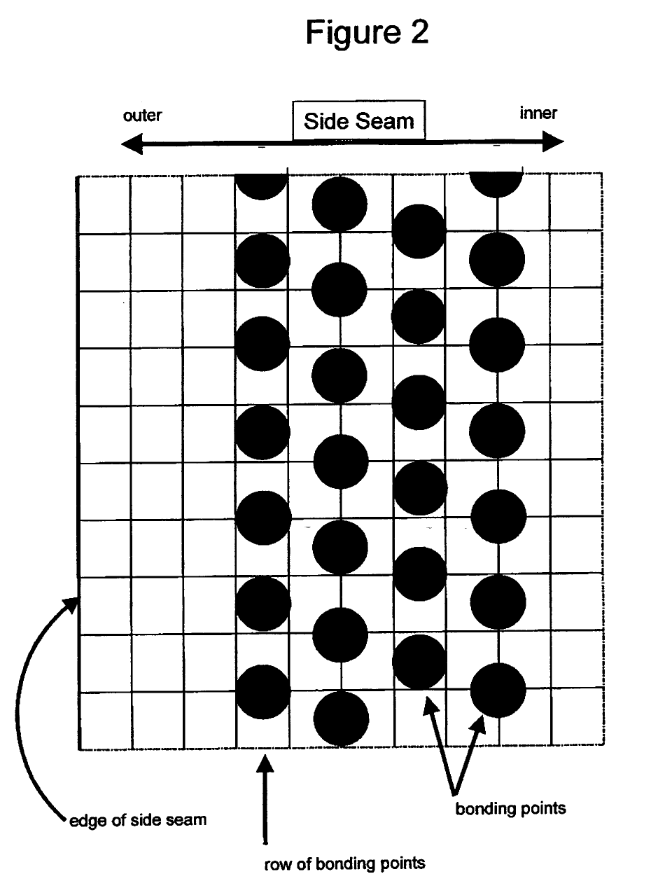

FIG. 2 illustrates a side seam bonding pattern 25 having improved softness and flexibility. In this configuration, the side seam bonding pattern contains four rows of circular bonding points. In this example, the outermost row is three millimeters from the edge of the article. This distance is referred to as the inset. It is contemplated that the inset may be other distances, such as any distance between about 0 mm to about 10 mm. In this illustration, the circular bonding points have a diameter of about 1 mm and are vertically spaced by about 0.5 mm. It is contemplated that the diameter of circular bonding points is any size, such as between about 0.2 mm to about 3.0 mm. It is further contemplated that the vertical spacing between bonding points may be any distance, such as between about 0.10 mm to about 3.0 mm. The vertical distance between bonding points need not be the same in any given row, nor must it be uniform across rows. Adjacent rows are shown as horizontally separated by about 0.5 mm. It is contemplated that the horizontal spacing between bonding points may be any distance, such as between about 0.10 mm to about 3.0 mm. The background grid, while not part of the bonding pattern, is included for reference and, in the illustrated embodiment, is scaled to 1 mm squares. In preferred embodiments, the width of the side seam, which is the width from the outermost edge of the side seam to the innermost edge of the innermost row of bonding points is less than about 25 mm, preferably less than about 15 mm, and more preferably less than about 10 mm.

In preferred embodiments, the bonding points of adjacent rows are offset from each other. FIG. 2 demonstrates an offset of 0.5 mm, or half the diameter of the circular bonding points, between adjacent rows. It is contemplated that the offset may be any convenient absolute distance or any convenient fraction of a proportion of a bonding point.

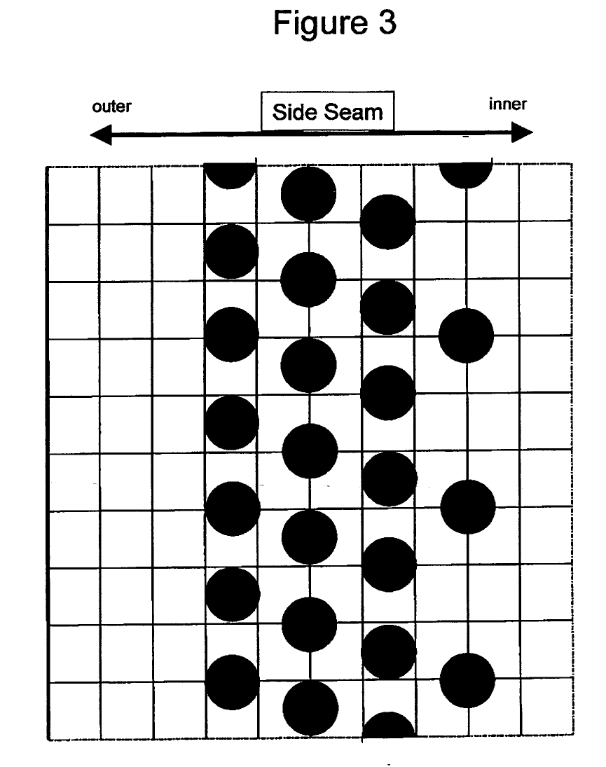

FIG. 3 illustrates an alternate configuration of a side seam bonding pattern 25 consistent with the principles described herein. FIG. 3 illustrates a side seam bonding pattern for which the innermost row has fewer bonding points than other, more external rows. Specifically, every other bonding point is omitted from the innermost row. This bonding pattern further improves the softness of the side seam against the wearer's skin. This side seam bonding pattern results in approximately a 33% bond area when the circular bonding points are 1 mm in diameter, vertically separated by 0.5 mm, and the rows are horizontally separated by 0.5 mm.

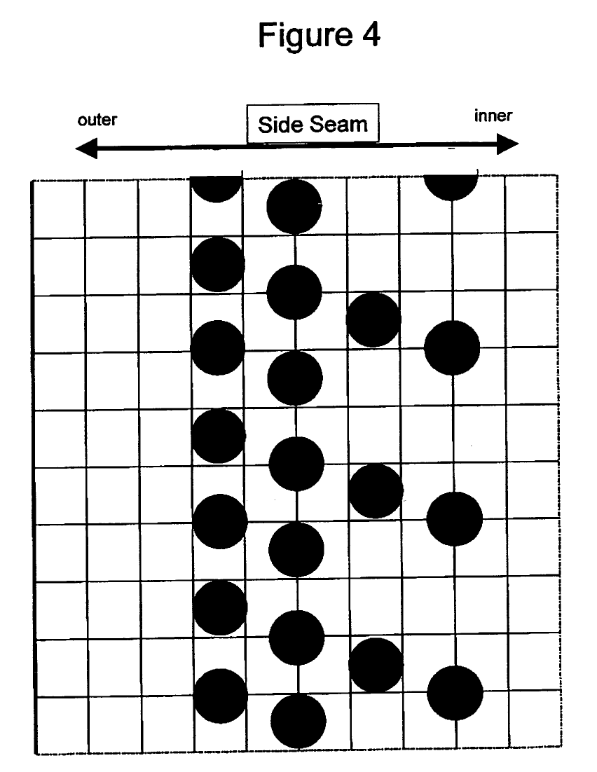

FIG. 4 illustrates another alternate configuration of a side seam bonding pattern 25 consistent with the principles described herein. In this embodiment, the innermost two rows have a reduced number of bonding points, similar to that described with regard to FIG. 3.

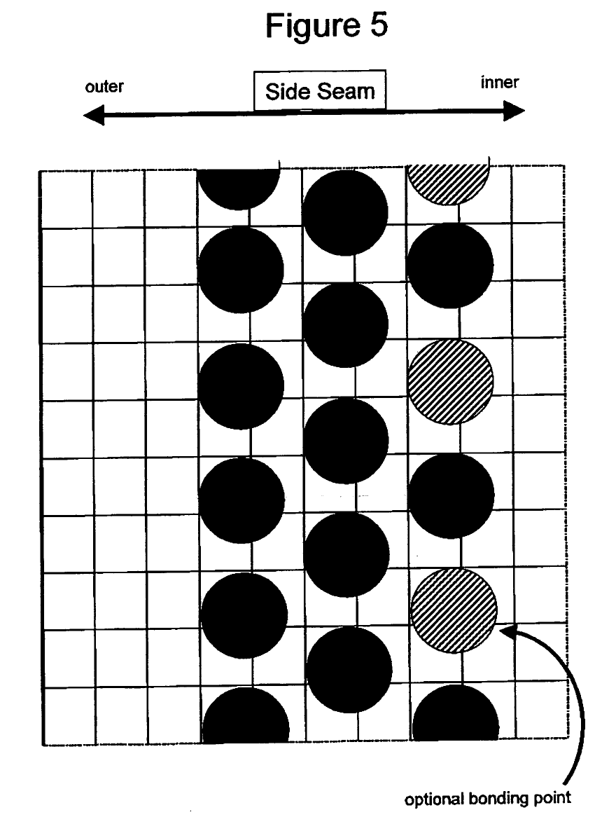

FIG. 5 illustrates another configuration of a side seam bonding pattern 25 consistent with the principles of this disclosure. The circular bonding points are aligned in three rows and illustrated as having a diameter of about 1.5 mm and the inset is 3 mm. The bonding points within a row are vertically offset by about 0.5 mm, and the rows are horizontally offset by about 0.5 mm. The innermost row optionally contains fewer bonding points than any of the other rows, where the hashed bonding points represent optional bonding points which may or may not be present. Side seams, having a bonding pattern with these dimensions and every other bonding point in the innermost row being omitted, have a bonding area of approximately 40%. The total width of the depicted side seam is 8.5 mm.

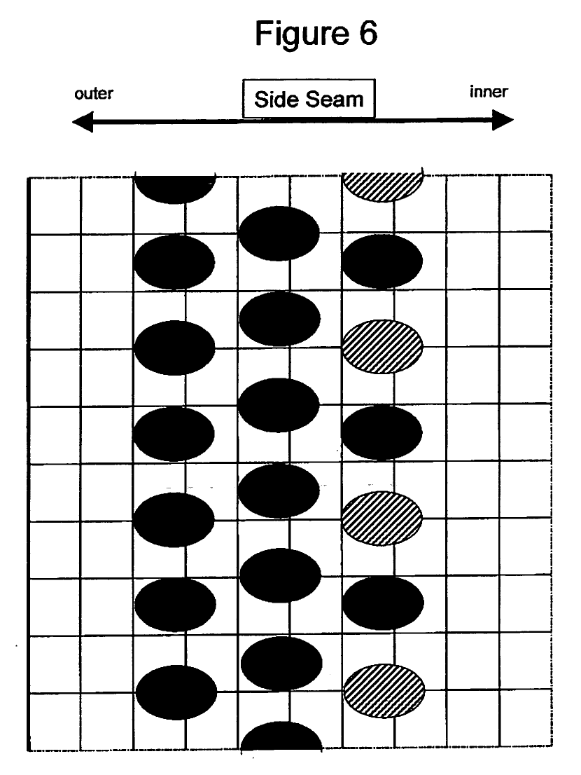

FIG. 6 illustrates another configuration of a side seam bonding pattern 25 having three rows of elliptical bonding points. The elliptical bonding points are about 1.5 mm along the long axis and about 1 mm along the short axis. The bonding points are vertically offset by about 0.5 mm within each row and the rows are horizontally offset by about 0.5 mm. Optionally, the innermost row has fewer bonding points than each of the other rows. This configuration is depicted as having an inset of about 2 mm. The hashed ellipses represent optional bonding points which may or may not be present. Other dimensions of side seam bonding patterns using elliptical bonding points are contemplated to fall within the scope of this disclosure.

The softness and flexibility of the side seam bonding patterns exemplified in FIGS. 2-6 benefit from several advantages. Bonding points having the shape of circles, ovals, or ellipses do not have the sharp edges and/or corners of rectangular or square point points and are, therefore, softer on the wearer's skin. The bonding point offset between adjacent rows of bonding points facilitates side seam folding along an axis perpendicular to the side seam. The plurality of rows of bonding points also facilitates folding along an axis parallel to the side seam, which increases flexibility compared to side seams having a single row of bonding points. Further, the bonding point offset between adjacent rows maintains or increases the tear strength across the side seam compared to side seams having a single row of bonding points because a rupture in a direction exactly perpendicular to the side seam is prevented.

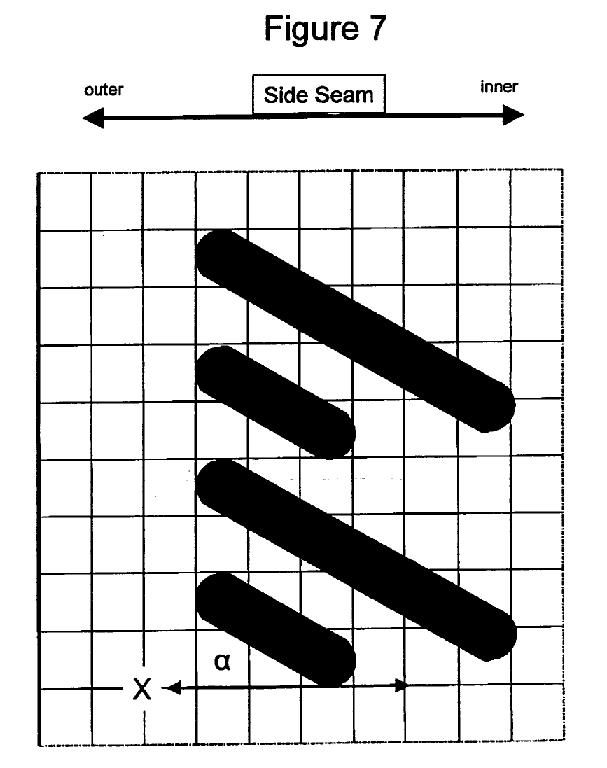

FIG. 7 illustrates another configuration of a side seam bonding pattern 25 using rounded end bar-shaped bonding points in a single row. The rounded end bar-shaped bonding points are shown as having a width of about 1 mm and a length of about 3 mm for the short bars and about 6 mm for the long bars. The bonding points are disposed at an angle α of about 300 from the axis (X) perpendicular to the side seam. When this bonding pattern is used and the ovals are vertically offset by about 1.5 mm, a bond area of about 32% is achieved.

In the side seam configuration that uses a single row of rounded end bar-shaped bonding points, the bonding points may have the same (not shown) or different dimensions. In preferred embodiments, different dimensions are used, but the outermost edge of the bonding points are aligned as shown. It is contemplated, however, that the outermost edges are not aligned. One particularly useful configuration centers the shorter ovals beneath the longer ovals. Suitable oval-shaped bonding points are between about 1.5 mm and about 8 mm in length. When bar shapes of different lengths are used, all convenient sizes are contemplated within the scope of this disclosure; however, in preferred dimensions, the shorter bar is between about 0.2 and 0.8 times the length of the longer bar.

It is further contemplated that the angle α is between about 20° and about 60°, but preferably between about 30° and about 45°. It is also contemplated that the bonding points may have another convenient shape that approximates a bar. Such shapes include, without limitation, rectangles and elongated ellipses. It is further contemplated that bonding points having shapes that do not approximate a bar are interspersed among the rounded end bar-shaped bonding points. For example, it is contemplated that the side seam configuration consists of a single row of bonding points having shapes that alternate between rounded end bar-shapes disposed at an angle α of about 30° and circles.

Side seams having bonding points with an elongated shape (e.g., bars, ovals and ellipses) disposed at an angle α to the plane perpendicular to the side seam have increased flexibility and softness. Disposition of the bonding points at an angle α facilitates the folding of the side seam along the axis parallel to the side seam while maintaining the ability of the side seam to be folded along the axis perpendicular to the side seam.

For any of the foregoing examples, it is apparent that a stronger side seam can be obtained by increasing the bond area. This may be achieved by using bonding points having a larger area, reducing the vertical distance between the bonding points within each row, reducing the horizontal distance between each row, and/or forming additional rows of bonding points.

The side seams and side seam bonding patterns described in this disclosure may be formed by any appropriate method. Methods known in the art for making side seams on absorbent articles include, for example, thermal bonding (i.e., melting) and ultrasonic bonding. Thermal bonding, for example, is done using a pair of rollers, of which one or both are heated. One roller is patterned with the desired bonding pattern and is apposed to an anvil roller which provides a smooth surface. The coverstock of the garment-facing surface and the body-facing surface are passed between the heated rollers and are thermally bonded.

Fluid permeable top sheets suitable for use with disposable absorbent articles similar to those of the present disclosure are well known in the art. The top sheet is sufficiently pervious to permit liquids (e.g., urine) to readily penetrate. Suitable top sheets may be woven or nonwoven materials, polymeric materials (e.g., apertured formed thermoplastic films, apertured plastic films, and hydroformed thermoplastic films), porous foams, reticulated foams, reticulated thermoplastic films, and thermoplastic scrims. Suitable woven and nonwoven materials include natural fibers (e.g., wood and cotton fibers), synthetic fibers (e.g., polymeric fibers such as polyester, polypropylene, and polyethylene fibers). In other embodiments, the top sheet may be treated with a surfactant to facilitate the transfer of fluid through the top sheet into the absorbent core.

Absorbent core materials and configurations for use with disposable absorbent articles similar to those of the present disclosure are also well known in the art. The absorbent core may be configured as a single continuous core or several discontinuous cores that may or may not be connected in fluid contact. The absorbent core may be symmetrical or asymmetrical and, specifically, may be configured to be gender specific. The shape of the absorbent core will depend upon the specific material used, the specific application for which the article is used, and the desired performance characteristics. The absorbent core(s) may consist of either a single type of absorbent material or may contain multiple types of absorbent materials. Suitable materials for the absorbent core include, for example, wood pulp, cellulose wadding, cotton fibers, absorbent foams, sponges, superabsorbent polymers (e.g., hydrogels), absorbent gelling materials, or combinations thereof.

Fluid impermeable back sheets for use with disposable absorbent articles similar to those of the present disclosure are also well known in the art. Preferably, the back sheet is a relatively thin (e.g., about 0.012 mm to about 0.051) plastic film. It is particularly desirable to use back sheets which, while being impervious to aqueous fluids, allows vapors to escape. Suitable material for such back sheets include, for example, microporous polyethylene and polypropylene films. In other configurations, the back sheet may be a laminate consisting of a fluid impervious film and a woven or nonwoven outer cover. There is no requirement that the outer cover be fluid impervious. Frequently the outer cover is selected for its aesthetic properties.

The disposable absorbent article of this disclosure may include structure to fasten the front and back side panels around the torso of the wearer. Such structure may include, for example, hooks, snap fasteners, and unilateral or bilateral external adhesive tabs. Alternatively, an adhesive may be applied directly to the inside face (e.g., directly on the body facing top sheet or by way of an adhesive-containing strip affixed to the top sheet) of either the front or back side panel. The adhesive is exposed by removing a protective film covering and the inner face of the adhesive-containing side panel is adhered to the outer face (back sheet) of the opposite side panel to form a side seam.

The assembly of the absorbent article may be performed according to well known principles in the art and may be varied depending upon the desired performance characteristics. For example, the absorbent cores may be “free floating” between the top sheet and back sheet, it may be adhered to the back sheet, or it may be elastically supported. The top sheet and back sheet may be joined by, for example, an adhesive or heat sealing (melting) the materials together. This process encapsulates the absorbent cores and all of the elastic members within the absorbent article to provide a unitary structure that is aesthetically appealing.

Although the foregoing disclosure has been described in some detail by way of illustration and example for purposes of clarity of understanding, it will be readily apparent to those of ordinary skill in the art in light of the teachings of this present disclosure that certain changes and modifications may be made thereto without departing from the spirit or scope of the appended claims.

What is claimed is:

1. An absorbent article comprising an absorbent core disposed between a body-facing surface and a garment-facing surface, wherein a side seam is formed by joining said opposing lateral edges of the body-facing surface of the front waist area of the article with the opposing lateral edges of the rear waist area with a bonding pattern, wherein said bonding pattern comprises a plurality of rows of bonding points along said side seam, wherein said rows are disposed parallel with said side seam.

2. The absorbent article of claim 1, wherein said bonding points are selected from the group consisting of circles, ovals, or ellipses, or a combination thereof.

3. The absorbent article of claim 1, wherein said bonding points are circles.

4. The absorbent article of claim 1, wherein said bonding pattern comprises three to five rows of bonding points.

5. The absorbent article of claim 3, wherein said bonding points are circles and separated in said rows by a distance equal to about 0.10 to about 1.25 times the diameter of said circles.

6. The absorbent article of claim 3, wherein said bonding points are circles having a diameter of about 0.50 mm to about 2.0 mm.

7. The absorbent article of claim 6, wherein said bonding points are offset in adjacent rows and said offset is equal to about 0.25 to about 0.75 times said diameter.

8. The absorbent article of claim 3, wherein at least one row comprises fewer bonding points as the laterally adjacent row.

9. The absorbent article of claim 8, wherein said row comprises about 0.25 to about 0.75 times as many bonding points as said laterally adjacent row.

10. The absorbent article of claim 9, wherein said row is the innermost row of said side seam.

11. The absorbent article of claim 1, wherein said bonding pattern comprises bonding points having a first shape and bonding points having a second shape, wherein said first shape is different from said second shape.

12. The absorbent article of claim 1, wherein said bonding pattern comprises bonding points having a first size and bonding points having a second size, wherein said first size is different from said second size.

13. An absorbent article comprising an absorbent core disposed between a body-facing surface and a garment-facing surface, wherein a side seam is formed by joining said body-facing surface and said garment-facing surface in a bonding pattern, wherein said bonding pattern comprises a row of bonding points along said side seam, wherein said row is disposed parallel with said side seam and said bonding points have an rounded end bar-shaped shape, and wherein said rounded end bar-shaped shape is disposed at an angle of between about 20° to about 60° from an axis perpendicular to said side seam.

14. The absorbent article of claim 13, wherein said bonding pattern comprises bonding points having substantially the same size.

15. The absorbent article of claim 13, wherein said bonding pattern comprises bonding points have different sizes.

16. The absorbent article of claim 13, wherein said bonding pattern comprises bonding points having a first oval length and bonding points having a second rounded end bar-shaped length, wherein said second rounded end bar-shaped length is between about 0.25 and about 0.75 times said first rounded end bar-shaped length.

17. The absorbent article of claim 16, wherein said bonding pattern is characterized by adjacent bonding points having rounded end bar-shaped lengths alternating between said first rounded end bar-shaped length and said second rounded end bar-shaped length.

18. The absorbent article of claim 13, wherein said angle is between about 300 and about 45°.
